(19) United States
(12) Patent Application Publication
(10) Pub. No.: US 2007/0025995 A1
(43) Pub. Date: Feb. 1, 2007

(54) Taxoid derivative covalently linked to tumor-specific antibody and a method for preparing the same

(76) Inventors: Hiroki Hamada, Naha-shi (JP); Katsuhiko Mikuni, Yokohama-shi (JP); Katsuyoshi Nakanishi, Yokohama-shi (JP); Tadakatsu Mandai, Kurashiki-shi (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/219,686

(22) Filed: Sep. 7, 2005

(30) Foreign Application Priority Data
Aug. 1, 2005 (JP) 2005-222617

(51) Int. Cl. A61K 39/395; C07K 16/46

(52) U.S. Cl. 424/155.1; 424/178.1; 530/388.8; 530/391.1; 536/17.1

(57) ABSTRACT

To provide a taxoid derivative that has improved solubility in water and acts specifically with respect to tumors, thereby mitigating side effects. The present invention provides an anticancer agent comprises covalent bond compound of an antibody that reacts specifically with respect to cancer cells with a taxoid derivative and a method for preparing the anticancer agent according to claim 1, characterized by reacting, in the presence of reduction agent, an antibody that reacts specifically with respect to cancer cells with a reaction product obtained by allowing periodic acid or periodate to act on a taxoid derivative.

### FIELD OF THE INVENTION

The present invention relates to an anticancer agent with few side effects that acts specifically on cancer cells, and a method for preparing the same. Specifically, the present invention relates to an anticancer agent comprises covalent bond compound of an antibody that reacts specifically with respect to cancer cells with a taxoid derivative and a method for preparing the anticancer agent.

### DESCRIPTION OF THE BACKGROUND

Paclitaxel (product name: Taxol) is a diterpene compound that is isolated from the bark, of a kind of Yew tree (namely, Taxus brevifolia) (M. C. Wani et al.: J. Am. Chem. Soc. 93, 2325 (1971)), and is known as a powerful anticancer agent having improvement effects with respect to cancers that could not be cured by any conventional chemotherapeutic methods. The mechanism of the anticancer action of paclitaxel is specific, and inhibits mitosis by inducing the overproduction of microtubules.

However, because paclitaxel is poorly soluble in water, its use as a cancer therapeutic agent is restricted. In order to improve solubility, solubilization agents have been used in paclitaxel preparations, but there have been problems with side effects induced by these solubilization agents.

For this reason, paclitaxel derivatives with increased solubility and preparations thereof have been developed (JP 9-286794 A), but these derivatives do not act specifically on cancer cells, and it is unavoidable to generate side effect. Accordingly, the mitigation of the side effects has become a problem.

### SUMMARTY OF THE INVENTION

The objective of the present invention, in light of the above state of affairs, is to develop a taxoid derivative that has high water solubility and acts specifically on cancer cells, thereby providing an effective anticancer agent.

The inventors of the present invention, as a result of painstaking investigations towards the development of a cancer therapeutic agent that acts specifically on cancer cells and has high solubility in water, succeeded in binding an antibody that is specific for cancer cells with a taxoid derivative having sugars.

This cancer therapeutic agent has high solubility in water, and in addition, because the bioavailability of the anticancer agent is increased by its specific action on cancer cells, few side effects and high therapeutic effects are expected to be attained. The inventors completed the present invention based on these findings.

Specifically, the present invention according to claim 1 is an anticancer agent comprises covalent bond compound of an antibody that reacts specifically with respect to cancer cells with a taxoid derivative.

The present invention according to claim 2 is the anticancer agent according to claim 1, wherein the antibody reacts any one of cells selected from the group consisting of ovarian cancer cells, breast cancer cells, lung cancer cells, pancreatic cancer cells and stomach cancer cells.

In addition, the present invention according to claim 3 is the anticancer agent according to claim 1 or 2, wherein the taxoid derivative is a compound having a taxane skeleton and sugars.

The present invention according to claim 4 is the anticancer agent according to any one of claims 1 to 3, wherein the taxoid derivatives are selected from the group consisting of 7-glucosyl acetyloxy paclitaxel, 2′-glucosyl acetyloxy paclitaxel, 7-maltooligosyl acetyloxy paclitaxel, 10-glucosyl acetyloxy paclitaxel, 10-glucosyl acetyloxy docetaxel, 10-galactosyl acetyloxy docetaxel and 10-mannosyl acetyloxy docetaxel.

Further, the present invention according to claim 5 is a method for preparing the anticancer agent according to claim 1, characterized by reacting, in the presence of reduction agent, an antibody that reacts specifically with respect to cancer cells with a reaction product obtained by allowing periodic acid or periodate to act on a taxoid derivative.

The present invention according to claim 6 is the method for preparing the anticancer agent according to any one of claims 5, wherein the taxoid derivatives are one selected from the group consisting of 7-glucosyl acetyloxy paclitaxel, 2′-glucosyl acetyloxy paclitaxel, 7-maltooligosyl acetyloxy paclitaxel, 10-glucosyl acetyloxy paclitaxel, 10-glucosyl acetyloxy docetaxel, 10-galactosyl acetyloxy docetaxel and 10-mannosyl acetyloxy docetaxel.

According to the present invention, it provides an anticancer agent in which an antibody that reacts specifically with respect to cancer cells is bound to a taxoid derivative, and a method for preparing the anticancer agent. The anticancer agent is provided with both high water solubility and properties of acting specifically on cancer cells. Therefore, the present invention realizes a cancer therapy that is extremely effective and places little burden on the patient in terms of side effects.

### BRIEF DESCRIPTION OF THE DRAWINGS

FIG. 1 shows the results of the measurement of tubulin polymerization activity in Test Example 1.

FIG. 2 shows the results of the measurement of anti-cancer action in Test Example 2.

FIG. 3 shows the results of the measurement of anti-cancer action in Test Example 3.

### DETAILED DESCRIPTION OF THE INVENTION

Hereinafter, the present invention will be described in detail.

The anticancer agent of the present invention is a product obtained by binding antibody that reacts specifically with respect to tumors to a taxoid derivative.

The taxoid derivative used in the present invention is a derivative in which sugars are bound via a spacer to paclitaxel. Examples of paclitaxel used include, beside the one obtained by isolation from the bark, branches or leaves of the Yew (Taxus brevifolia), according to a method described in Kingston, D. G. I.: Pharmacol. Ther., 52, 1 (1992), chemically synthesized compounds (R. A. Holton: European Patent-A 400971, 1990) etc. As a sugar, in addition glucose, mannose and galactose may be used.

Taxoid derivatives can be prepared by the method described in Japanese Patent Laid-open No. Hei 9-286794. Specifically, the derivative is obtained by binding a spacer (glycolate such as ethylene glycolate) to tetrabenzylglucose, which is obtained by a conventional method using glucose as the starting substance, thereby producing an ester compound, whereupon deethylation is performed to obtain a tetrabenzyl acetic acid oxiglucoside, and this substance is then allowed to react with paclitaxel. The target compound can also be obtained by a similar reaction in cases where a sugar other than glucose is used.

Examples of taxoid derivatives that may be cited include 7-glucosyl acetyloxy paclitaxel (hereinafter sometimes referred to 7-GLG-PT), 2′-glucosyl acetyloxy paclitaxel (hereinafter sometimes referred to 2′-GLG-PT), 7-maltooligosyl acetyloxy paclitaxel, 10-glucosyl acetyloxy paclitaxel (hereinafter sometimes referred to 10-GLG-PT), 10-glucosyl acetyloxy docetaxel, 10-galactosyl acetyloxy docetaxel and 10-mannosyl acetyloxy docetaxel. Among them, 7-GLG-PT is especially preferable.

A preferable example of an antibody that reacts specifically with respect to cancer cells used in the present invention is monoclonal antibody directed against cells of human ovarian cancer, breast cancer, lung cancer, pancreatic cancer and stomach cancer_cells. The antibody that reacts specifically with respect to these cancer cells may be produced by inoculating an appropriate host such as mouse, rat, Guinea pig or rabbit with the above cancer cells, and then collecting the resulting antibody from body fluid. Commercially available products may also be used.

According to the present invention, the reaction, whereby the taxoid derivative and antibody that reacts specifically with respect to cancer cells are covalently bound, is explained. First, a reaction product is obtained by a selective oxidation reaction wherein a taxoid derivative is allowed to react with periodic acid or periodate, such as sodium periodate solution. This reaction involves a step of converting a sugar moiety of the taxoid derivative to its aldehyde form, which is then bonded to an amino group on the antibody. As periodate, in addition to sodium periodate, potassium periodate, sodium metaperiodate, and potassium metaperiodate can be used.

The reaction is carried out at room temperature between 0 to 40° C. for 30 minutes to 5 hours, preferably for 1 to 2 hours. Upon completion of the reaction, the insoluble matter is removed by a solid-liquid separation means such as centrifugation. In addition, the excess sodium periodate is removed by passing the reaction solution through a desalting column (e.g., D-Salt Polyacrylamide 1800 Desalting Column; manufactured by Pierce). Alternatively, in stead of desaltation, it is possible to terminate the reaction by adding ethylenglycol, thereby proceeding to next step.

Next, by reacting antibody and the above reaction product in the presence of reduction agent to bond them together, the anticancer agent (antitumor agent) of the present invention is obtained. As reduction agent, sodium cyanoborohydride, sodium borohydride and so forth can be used.

The reaction is carried out for 1 to 30 hours, preferably 10 to 20 hours, at room temperature between 0 to 40° C. by adding antibody dissolved in phosphate buffer and sodium cyanoborohydride to the above reaction product (desalted solution). Upon completion of the reaction, centrifugation is carried out using centrifugal concentration membrane in order to remove the low molecular weight compounds, thereby obtaining the target substance having the anticancer action.

The substance having the anticancer action (the antitumor action) of the present invention obtained in this manner is a clear transparent liquid. This substance can be used as it is in order to prepare an anticancer agent, but a powder can be produced, as necessary, by lyophilization or spray-drying, or, alternatively, the substance may be used in a configuration such as a tablet or microcapsule. When this substance is used for formulation, the examples of dosage form are not limited to but may include solutions, powders, tablets and microcapsules. At this time, components that are commonly used may be added appropriately as desired, such as carriers, excipients, extending agents, sweeteners, fragrance, emulsifiers and other additives that are commonly used in the field of pharmaceutics.

The binding product of taxoid derivative and antibody (e.g., 7-GLG-PT), which is the anticancer agent of the present invention, has improved solubility with respect to water, and, whereas the solubility of paclitaxel is 0.4 μg/mL, the solubility of anticancer agent of 7-GLG-PT used in Example 1 herein below, for example, is 22.6 μg/mL.

The anticancer agent of the present invention has the structure of paclitaxel or docetaxel, and thus it was conjectured that the compound manifests its anticancer effects by inducing overproduction of tubulin in the same manner as paclitaxel or docetaxel. However, this compound blocks tubulin polymerization in the same manner as vinblastine. The tubulin polymerization activity assay was, as shown in Test Example 1, determined by measuring the absorbance at 340 nm and measuring tubulin polymerization activity.

The in vitro evaluation method of Test Example 2 is a method in which cancer cells are cultured and the cell number is determined calorimetrically.

According to the present invention, an anticancer agent having few side effects and high water solubility, as well as a method for preparing thereof, are provided. The anticancer agent of the present invention can be made to act effectively and selectively with respect to various cancers by the appropriate selection of the antibody that is bound to the taxoid derivative.

Consequently, the present invention contributes to the field of chemotherapeutics for various cancers.

### EXAMPLES

The present invention is described in more detail below by referring to Examples, but the present invention is not limited thereto.

### Example 1

150 μg of 7-GLG-PT was dissolved in 6.25 mL of a 0.3 M sodium periodate solution to react for 2 hours at room temperature. Upon completion of the reaction, centrifugation was carried out for 10 minutes at 15,000 rpm in order to remove insoluble matter from the reaction solution.

Next, the reaction solution was passed through a desalting column (product name: D-Salt Polyacrylamide 1800 Desalting Column; manufactured by Pierce) in order to remove the excess sodium periodate.

To the resulting desalted reaction solution, added were 2 mL of 1 M phosphate buffer (pH 7.0), 1 mL of antibody solution (product name Monoclonal Mouse Anti-Human Antibody CA125, Clone OC125, protein concentration 5.6 mg/mL; ovarian tumors antibody; manufactured by Dako) and 160 mg of sodium cyanoborohydride, and the reaction was made for 16 hours at room temperature.

Upon completion of the reaction, the reaction solution was centrifuged for 15 minutes at 5000 rpm using centrifugal concentration membrane devices (molecular weight cut-off 30,000 Da; manufactured by Amicon).

A total of 3 mL of reaction solution (product of the present invention; CA125DV) was obtained in this manner.

### Test Example 1

Tubulin polymerization activity was measured using the HTS-Tubulin Polymerization Assay Kit (manufactured by Cytoskeleton) with respect to both the reaction solution of the present invention obtained in Example 1 and 3.7 mg/mL aqueous solution of paclitaxel used as a control.

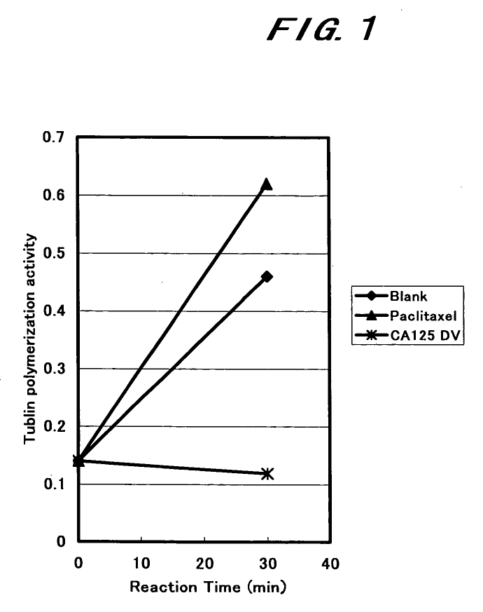

The results are shown in FIG. 1. The horizontal axis in the figure denotes reaction time (min) and the vertical axis represents tubulin polymerization activity (OD340). From FIG. 1, it is clear that tubulin polymerization was stimulated by paclitaxel, whereas tubulin polymerization was inhibited by the product of the present invention (CA125DV).

Therefore, it is clear that the product of the present invention has anticancer action.

### Test Example 2

The final reaction solution obtained in Example 1 was lyophilized to obtain a powder of the product of the present invention. This powder was dissolved in distilled water at room temperature to produce a concentration of 100 mg/mL, and the powder dissolved completely.

Meanwhile, human-derived ovarian tumors cells SHIN-3, KOC-2s were cultured on RPMI1640 media (manufactured by Nissui, Tokyo) containing 10% FCS under 5% CO₂ at 37° C., and then were inoculated into a 96-well plate (manufactured by Corning Incorporated, Corning, N.Y.) at 10⁴ cells per well.

1 day after the inoculation, aqueous solution of the anticancer agent of the present invention (CA125DV) was added to each well at a final concentration of 10 mg/mL or 5 mg/mL. After 24 hours, MTT (3-(4,5-dimethylthiazol-2-yl)-2,5-diphenyltetrazolium bromide) was added, and the supernatant was then aspirated after 4 hours. DMSO was added and the absorbance at 570 nm was measured.

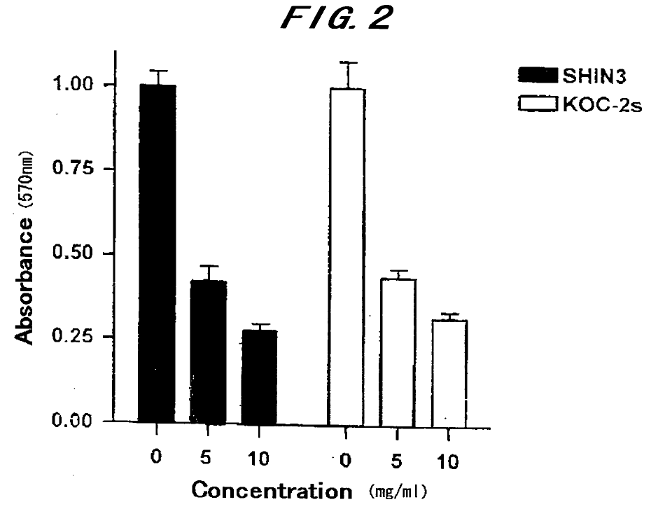

The results are shown in FIG. 2. In the figure, the vertical axis represents absorbance (570 nm), and the horizontal axis represents anticancer agent concentration. From these results, it is clear that the anticancer agent of the present invention has anticancer activity even in cell systems.

### Example 2

150 μg of 7-oligosyl acetyloxy-PT was dissolved in 6.25 mL of a 0.3 M sodium periodate solution to react for 2 hours at room temperature. Upon completion of the reaction, centrifugation was carried out for 10 minutes at 15,000 rpm in order to remove insoluble matter from the reaction solution.

Next, the reaction solution was passed through a desalting column (product name: D-Salt Polyacrylamide 1800 Desalting Column; manufactured by Pierce) in order to remove the excess sodium periodate.

To the resulting desalted reaction solution, added were 2 mL of 1 M phosphate buffer (pH 7.0), 1 mL of antibody solution (product name Monoclonal Mouse Anti-Human Antibody CA125, Clone OC125, protein concentration 5.6 mg/mL; ovarian tumors antibody; manufactured by DAKO) and 160 mg of sodium cyanoborohydride, and the reaction was carried out for 16 hours at room temperature. Upon completion of the reaction, the reaction solution was centrifuged for 15 min at 5000 rpm using centrifugal concentration membrane devices (molecular weight cut-off 30,000 Da; manufactured by Amicon).

A total of 3 mL of reaction solution (product of the present invention; CA125DV) was obtained in this manner.

### Test Example 3

The final reaction solution obtained in Example 2 was lyophilized to obtain a powder of the product of the present invention. This powder was dissolved in distilled water at room temperature to produce a concentration of 100 mg/mL, and the powder dissolved completely. Meanwhile, human-derived ovarian tumors cells SHIN-3, KOC-2s were cultured on RPMI1640 media (manufactured by Nissui, Tokyo) containing 10% FCS under 5% CO₂ at 37° C., and then were inoculated into a 96-well plate (manufactured by Corning Incorporated, Corning, N.Y.) at 10⁴ cells per well.

1 day after the inoculation, aqueous solution of the anticancer agent of the present invention (CA125DV) was added to each well at a final concentration of 10 mg/mL or 5 mg/mL. After 24 hours, MTT (3-(4,5-dimethylthiazol-2-yl)-2,5-diphenyltetrazolium bromide) was added, and the supernatant was then aspirated after 4 hours. DMSO was added and the absorbance at 570 nm was measured.

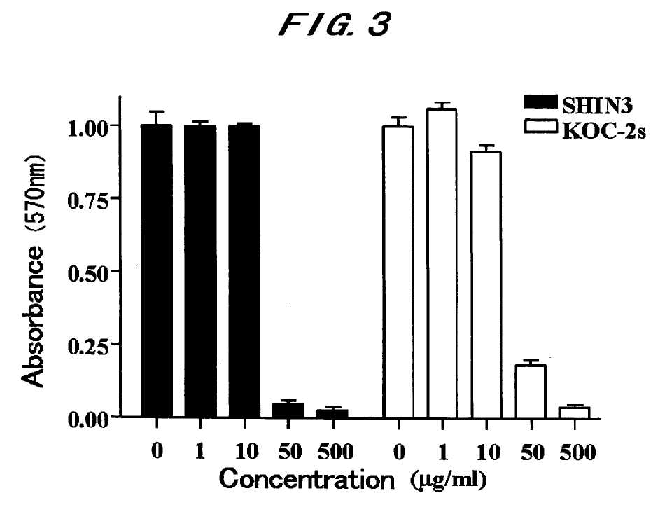

The results are shown in FIG. 3. In the figure, the vertical axis represents absorbance (570 nm), and the horizontal axis represents anticancer agent concentration. From these results, it is clear that the anticancer agent of the present invention has anticancer activity even in cell systems.

1. An anticancer agent comprises covalent bond compound of an antibody that reacts specifically with respect to cancer cells with a taxoid derivative.

2. The anticancer agent according to claim 1, wherein the antibody is an antibody that reacts specifically with any one of cells selected from the group consisting of ovarian cancer cells, breast cancer cells, lung cancer cells, pancreatic cancer cells and stomach cancer cells.

3. The anticancer agent according to claim 1, wherein the taxoid derivative is a compound having a taxane skeleton and sugars.

4. The anticancer agent according to claim 1, wherein the taxoid derivative is selected from the group consisting of 7-glucosyl acetyloxy paclitaxel, 2′-glucosyl acetyloxy paclitaxel, 7-maltooligosyl acetyloxy paclitaxel, 10-glucosyl acetyloxy paclitaxel, 10-glucosyl acetyloxy docetaxel, 10-galactosyl acetyloxy docetaxel and 10-mannosyl acetyloxy docetaxel.

5. A method for preparing the anticancer agent according to claim 1, characterized by reacting, in the presence of reduction agent, an antibody that reacts specifically with respect to cancer cells with a reaction product obtained by allowing periodic acid or periodate to act on a taxoid derivative.

6. The method for preparing the anticancer agent according to claim 5, wherein the taxoid derivative is selected from the group consisting of 7-glucosyl acetyloxy paclitaxel, 2′-glucosyl acetyloxy paclitaxel, 7-maltooligosyl acetyloxy paclitaxel, 10-glucosyl acetyloxy paclitaxel, 10-glucosyl acetyloxy docetaxel, 10-galactosyl acetyloxy docetaxel and 10-mannosyl acetyloxy docetaxel.

7. The anticancer agent according to claim 2, wherein the taxoid derivative is a compound having a taxane skeleton and sugars.

8. The anticancer agent according to claim 7, wherein the taxoid derivative is selected from the group consisting of 7-glucosyl acetyloxy paclitaxel, 2′-glucosyl acetyloxy paclitaxel, 7-maltooligosyl acetyloxy paclitaxel, 10-glucosyl acetyloxy paclitaxel, 10-glucosyl acetyloxy docetaxel, 10-galactosyl acetyloxy docetaxel and 10-mannosyl acetyloxy docetaxel.

9. The anticancer agent according to claim 3, wherein the taxoid derivative is selected from the group consisting of 7-glucosyl acetyloxy paclitaxel, 2′-glucosyl acetyloxy paclitaxel, 7-maltooligosyl acetyloxy paclitaxel, 10-glucosyl acetyloxy paclitaxel, 10-glucosyl acetyloxy docetaxel, 10-galactosyl acetyloxy docetaxel and 10-mannosyl acetyloxy docetaxel.

10. The anticancer agent according to claim 2, wherein the taxoid derivative is selected from the group consisting of 7-glucosyl acetyloxy paclitaxel, 2′-glucosyl acetyloxy paclitaxel, 7-maltooligosyl acetyloxy paclitaxel, 10-glucosyl acetyloxy paclitaxel, 10-glucosyl acetyloxy docetaxel, 10-galactosyl acetyloxy docetaxel and 10-mannosyl acetyloxy docetaxel.
